(19) United States
(12) Patent Application Publication
(10) Pub. No.: US 2012/0232451 A1
(43) Pub. Date: Sep. 13, 2012

(54) Pavlik Harness and Accessories Therefor

(76) Inventor: Christina Topinka, Universal City, TX (US)

(21) Appl. No.: 13/415,872

(22) Filed: Mar. 9, 2012

Related U.S. Application Data
(60) Provisional application No. 61/450,883, filed on Mar. 9, 2011.

(51) Int. Cl. A61F 5/00

(52) U.S. Cl. 602/19

(57) ABSTRACT

The invention in at least one embodiment includes a base having a plurality of spaced apart fastening components along two opposing edges, and the base having sufficient width and length to wrap around a portion of a harness for treating developmental dysplasia of the hip. The invention in another embodiment includes a cover that extends beyond the edges and the free end of a strap free end where the strap includes a hook section and a loop section and is part of a harness for treating developmental dysplasia of the hip. The invention in another embodiment includes a harness for treating developmental dysplasia of the hip combined with either of the other two embodiments.

This application claims the benefit of U.S. provisional Application Ser. No. 61/450,883, filed Mar. 9, 2011, which is hereby incorporated by reference.

### I. FIELD OF THE INVENTION

This invention relates to an elegant improvement and addition to Pavlik Harnesses that provides in at least one embodiment protection of an infant's skin from abrasions and/or cuts.

### II. BACKGROUND OF THE INVENTION

A variety of studies have found the incidence rate of developmental dysplasia of the hip is between 1 per 1000 to 55.1 per 1000. Viktor Bialik, “Developmental Dysplasia of the Hip: A New Approach to Incidence”, Pediatrics, Vol. 103, No. 1, pp. 93-99, January 1999. A common tool used to treat this condition in infants is a Pavlik Harness to see if this resolves the condition before deciding if closed reduction or open reduction surgery is required to treat the condition.

An example of a Pavlik Harness is disclosed in U.S. Pat. No. 4,913,136, which is hereby incorporated by reference. The Pavlik Harness disclosed includes a chest halter and two foot attachments (or stir-ups) each connected via two straps to the chest halter. The chest halter includes a belt around the chest and two shoulder straps. The leg and shoulder straps are adjustable using straps with a hook section and loop section on each shoulder strap and leg strap that loop through D-rings. The D-rings (or other ring or buckle although not disclosed in the patent) may be located on the chest belt and on the foot attachments (not disclosed).

The harness includes a variety of straps with hook and loop sections such as those offered by Velcro® to hold the harness in place on the infant. The strap includes a free end that typically includes a nylon material backing that has a sharp edge to it that will cause abrasions and cuts on the infant's skin. The abrasions and cuts occur particularly in the wrist area of newborns when the arms are held against the body with the wrists at about shoulder height. The injuries are further multiplied when the infants are swaddled by hospital staff and later the parents.

Typically the infant will wear the harness 24 hours a day for a period of weeks and if diagnosed at birth for the first two (2) months, which minimizes the clothing that can be used underneath the harness to cover the infant's body. To address this problem, it is known to use larger sized onesies over the infant's body and the harness to keep the infant warm. Sometimes the onesies are modified to increase the ease of access to change diapers. Otherwise the infant may wear the same clothing for a period of time if worn below the harness with changes occurring during adjustments or after the medical professionals have trained the parents on manipulating the harness.

When the diagnosis occurs at birth, an ultrasound is performed to check the growth and development of the hip socket in the infant around one (1) month after birth and an X-ray is taken after about two (2) months due to hardening of the bones. If the hip is healing, then the harness is worn for approximately another month during naps and at bedtimes. Typically, at this point the parents are taught by the medical staff on how to remove the harness and to place the harness back onto the infant, which allows the infant to be fully bathed at home instead of just receiving a sponge bath.

A further problem caused by the harness occurs during breast feeding. Just as the sharp edges of the hook and loop sections irritate and cut the infant's skin, these same sections also can cut and injury the mother's breasts along with causing scratches to develop from the edge of the Velcro harness sections, such as the hook sections and loop sections.

An additional problem that arises is that exposed hook and loop sections on the straps will at times catch or attach themselves to clothing, fabric seats, or fabric furniture along with collect lint and other debris.

### III. SUMMARY OF THE INVENTION

This invention in at least one embodiment provides protection for the infant's skin from injury resulting from the sharp edges of hook and loop strap.

At least one embodiment according to the invention includes a cover for use with a harness for treating developmental dysplasia of the hip, the harness having a chest belt with a pair of shoulder straps and buckles with each shoulder strap having a loop section and a hook section, the cover including a base with a front face, a back face, and two opposing edges; a plurality of snap pairs having a socket and a stud, one of each component of the snap pairs is attached to the front face along one edge of the base and the other components of the snap pairs are attached to the rear face along the opposing edge of the base, the snap components are distributed along each edge such that each aligned pair includes one socket and one stud; and wherein the base having sufficient width between the opposing edges to encircle one shoulder strap when the hook section is engaging the loop section.

At least one embodiment according to the invention includes a cover for use with a harness for treating developmental dysplasia of the hip, the harness having a chest belt with a pair of shoulder straps and buckles with each shoulder strap having a loop section and a hook section, the cover including a base with a front face, a back face, and two opposing edges; a plurality of means for fastening the front face to the back face along their respective opposing edges; and wherein the base having sufficient width between the opposing edges to encircle one shoulder strap when the hook section is engaging the loop section.

At least one embodiment according to the invention includes a harness for treating developmental dysplasia of the hip, the harness including a chest belt; at least two chest buckles attached directly to the chest belt and spaced from each other; and at least two shoulder straps extending from the chest belt, each shoulder strap capable of passing through one of the chest buckles, each shoulder strap having a free end with a hook section and a loop section, a cover attached to at least one of the hook section and the loop section, the cover extending beyond the sides of the strap, the cover having a front face, a back face, and two opposing edges spaced from the strap, means for fastening the front face to the back face along respective opposing edges and around the hook section and the loop section when the hook and loop section are engaging each other.

At least one embodiment according to the invention includes a harness for treating developmental dysplasia of the hip, the harness including a chest belt; at least two chest buckles attached directly to the chest belt and spaced from each other; and at least two shoulder straps extending from the chest belt, each shoulder strap capable of passing through one of the chest buckles, each shoulder strap having a free end with a hook section and a loop section, a cover attached to at least one of the hook section and the loop section, the cover extending beyond the free end and having a width wider than at least one of the hook section and the loop section.

At least one embodiment according to the invention includes the use of medical stockings on the infant's feet and legs below the harness worn by the infant. The harness in further embodiments is modified and/or used in conjunction with the previously identified embodiments.

Given the following enabling description of the drawings, the apparatus should become evident to a person of ordinary skill in the art.

### IV. BRIEF DESCRIPTION OF THE DRAWINGS

The present invention is described with reference to the accompanying drawings. In the drawings, like reference numbers indicate identical or functionally similar elements. The use of cross-hatching and shading within the drawings is not intended as limiting the type of materials that may be used to manufacture the invention. The use of a baby doll 130 and a stuffed bunny rabbit 140 in the figures is for illustration purposes. No claim or affiliation is asserted by use of a Rhino Kicker sold by Pediatric Orthopedic Designs, Inc. in the figures.

FIG. 1 illustrates a front and back views of an embodiment according to the invention.

FIG. 2 illustrates a front and back views of an alternative embodiment to that illustrated in FIG. 1.

FIG. 3 illustrates a front view of a harness with a button embodiment illustrated in FIG. 1 installed on the harness.

FIG. 4 illustrates a side view of the embodiment installed on a harness illustrated in FIG. 3.

FIG. 5 illustrates a rear/side view of the embodiment installed on a harness illustrated in FIG. 3.

FIG. 6 illustrates a rear view of the embodiment installed on a harness illustrated in FIG. 3.

FIG. 7 illustrates an alternative embodiment according to the invention.

FIG. 8 illustrates an alternative embodiment according to the invention.

FIG. 9 illustrates a front view of a harness with the embodiment illustrated in FIG. 8.

FIG. 10 illustrates a closer view of one of the shoulder straps from FIG. 9 with the embodiment of FIG. 8 attached.

FIG. 11 illustrates a rear view of the harness depicted in FIG. 9.

### V. DETAILED DESCRIPTION OF THE DRAWINGS

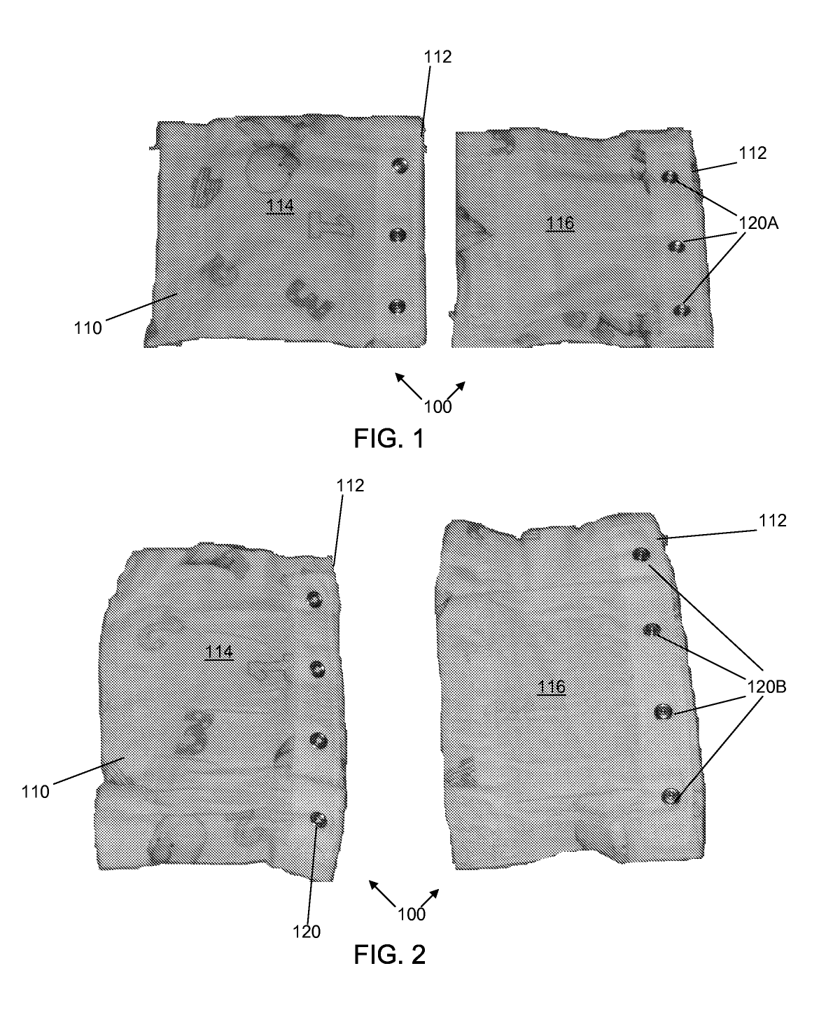

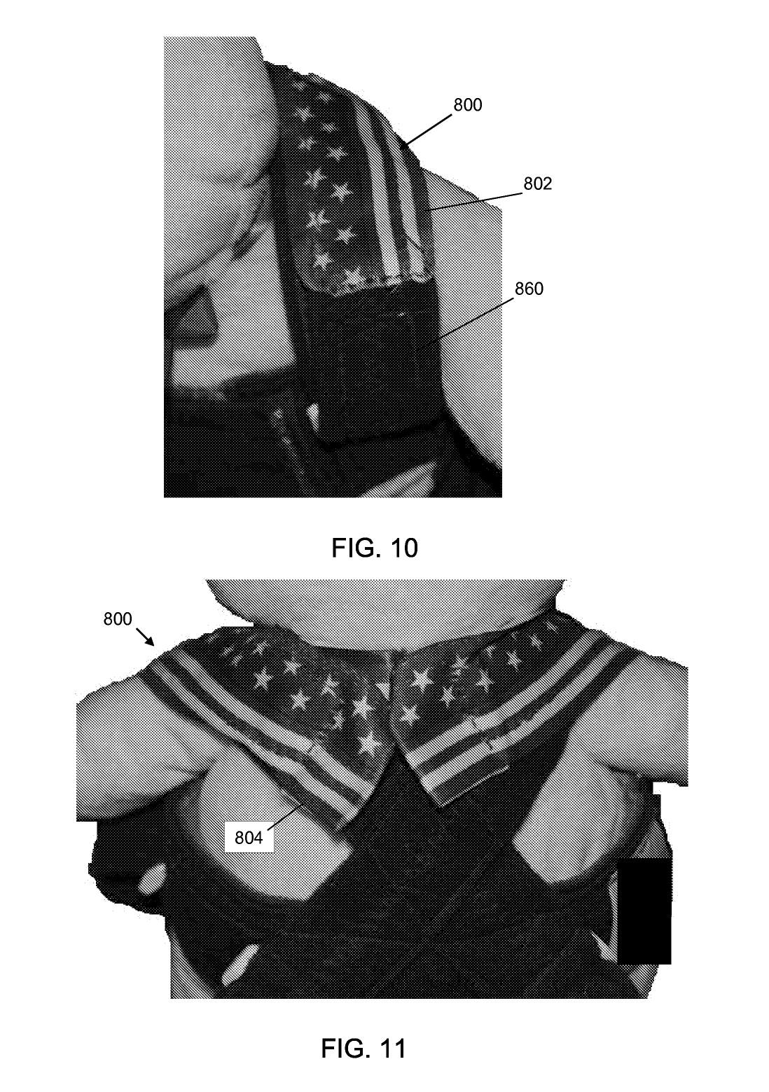

FIGS. 1-11 illustrate different embodiments according to the invention that decrease the risk of abrasions and cuts on infants wearing a Pavlik harness.

FIG. 1 illustrates one embodiment according to the invention. As illustrated, the embodiment includes a cover 100 with a base 110 and a plurality of snaps 120 running along two opposing edges 112 of the base 110.

The snaps 120 are orientated such that when the base 110 is wrapped around the engaged loop and hook sections 162, 164 of the harness strap 160 the snaps 120 snap together to hold the base 110 substantially in place. In some embodiments the hook and loop sections are intermingled such that both hooks and loops are present together along the strap, and as such reference to individual sections in this disclosure should be understood that instead of two separate discrete hook and loop sections there is one combination section of hooks and loops. As illustrated, half of the snap parts run down one edge 112 of the base 110 while the other half of the snaps run along the opposite edge 112 on the opposing face of the base such that each face (i.e., front (or top side) 114 and back (or bottom or under side) 116) of the of the base 110 has one row of snap components. The snap components are aligned to allow each respective pair of socket 120A and stud 120B to engage each offer to fasten together. FIG. 2 illustrates an alternative embodiment that includes four (4) snap pairs compared to three (3) snap pairs illustrated in FIG. 1. The number of snap pairs can vary from 2 to 6. In at least one embodiment the distance between snap components run along one edge is approximately 1.5 inches, but could be spaced in a range of 0.2 inches to 2.5 inches including the end points.

In at least one embodiment, the base 110 is made of fabric or cloth. Examples of fabric that may be used for the base include any lightweight fabric that in at least one embodiment is also soft such as flannel, Minky or other micro-fiber cloth, Modal alone or with spandex, Lyocell, cotton, Jersey, and Velor. As illustrated, the base 110 in at least one embodiment includes a piece of cloth that has its edges folded over and sewn to finish the edge. Although a pattern is illustrated as being present on the front face 114, the base 110 may be plain or have other patterns typical for fabrics for use in infant clothing, bedding, or pillows. The illustrated bases in FIGS. 1 and 2 have dimensions of approximately 4.25 inches by approximately 4.5 inches and approximately 4.0 inches by approximately 6.25 inches, respectively; however, the size of the cover 110 can be adjusted to reflect the size of the harness. After the edge is sewn, half of the snap pieces are sewn along one edge of the fabric or otherwise attached and the respective matching snap pieces remaining are attached along the opposite edge and opposite face of the base 110.

An alternative way to attach the snaps 120 is to use a strip of fabric (as illustrated in FIGS. 1 and 2) with the snaps 120 already attached to it and sew the snap strips along the opposing edges and faces of the cover 110. In the alternative way, the snap fabric is sewn around its periphery to attach it to the base 110. A further alternative embodiment is to replace the snaps 120 with other fasteners including, but not limited to, hook and loop circular tabs (for example, tabs sold by Velcro®), plastic hook enclosures, or buttons. These different fasteners are examples of means for fastening.

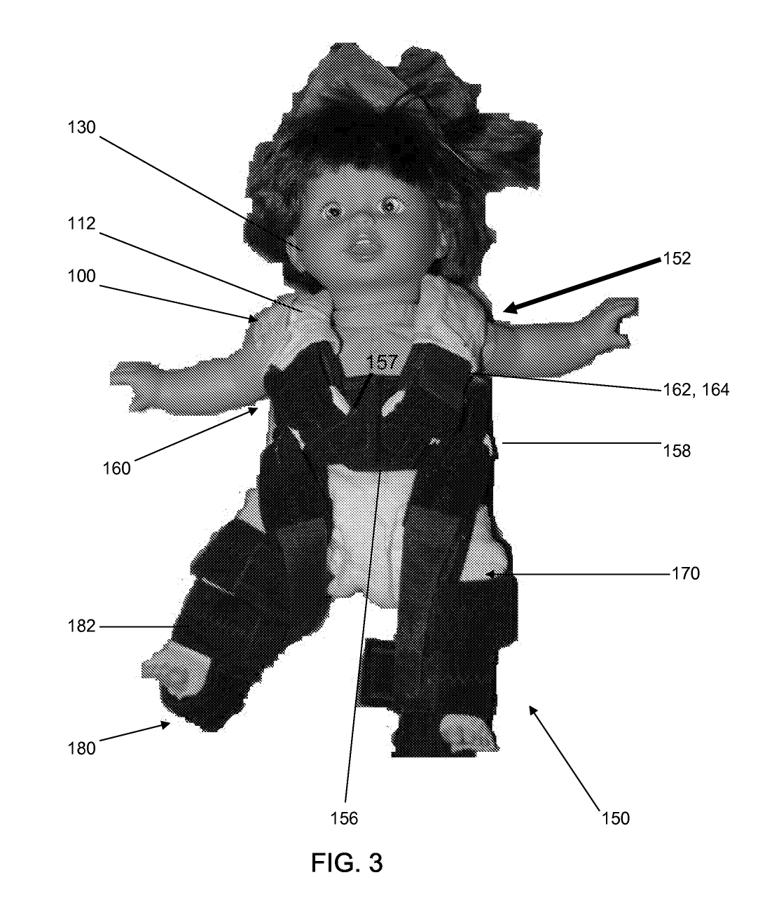

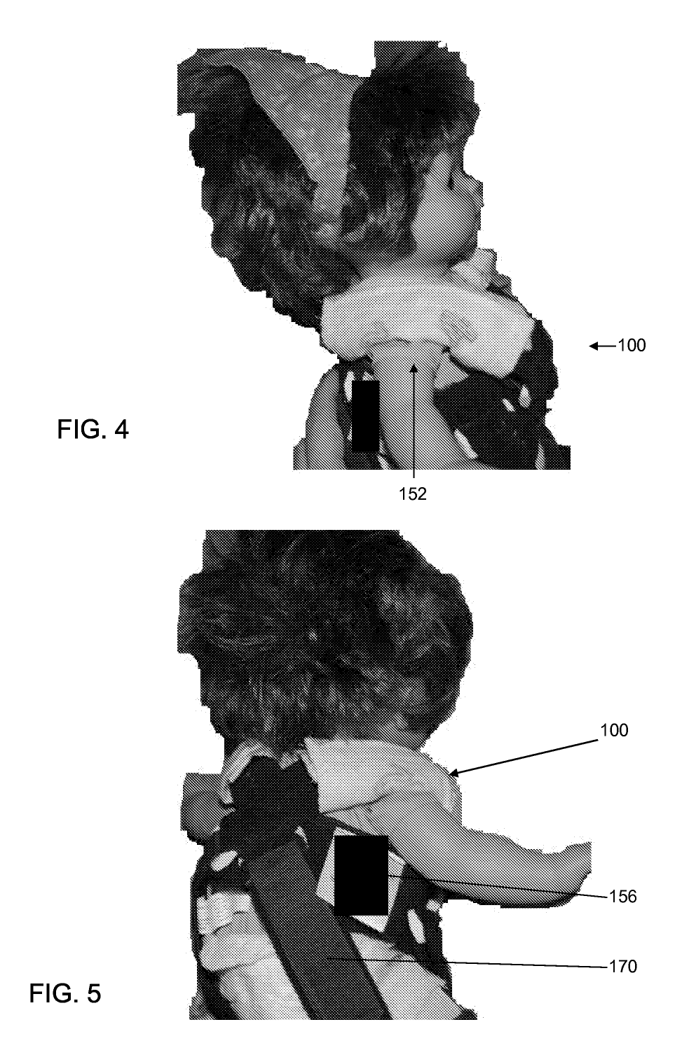

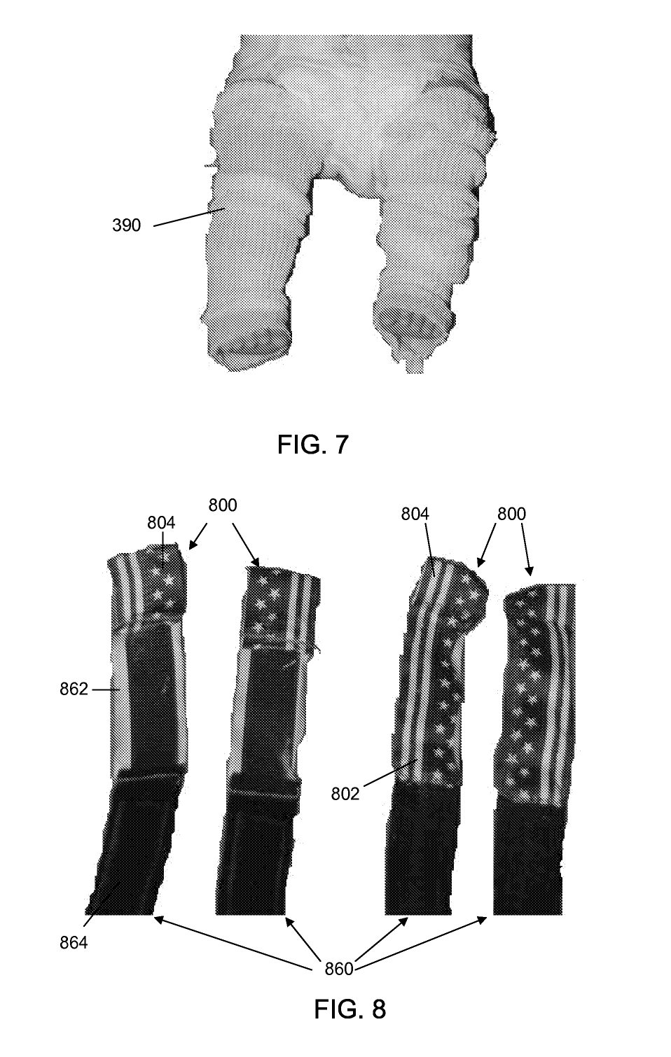

FIGS. 3-6 illustrate a doll 130 wearing a harness 150 with two (2) covers 100 installed on the shoulder section 152 of the harness 150 where the hook and loop sections 162, 164 are engaged. As illustrated in FIG. 4, the cover 100 has an orientation with the snap edge 112 facing up to reduce possible rubbing of the snap edge 112 against the infant's body. FIG. 3 also illustrates another embodiment of the invention that is better illustrated in FIG. 7, this embodiment includes gauze sleeves (or toeless stockings) 390 pulled up over the infant's feet and legs to reduce the shafting and rubbing of the infant's skin against the harness. Although not illustrated, it should be understood from this disclosure that the cover 100 could also be installed over the straps 170 used for the foot stirrups 180.

In an alternative embodiment, the cover 100 includes padding that is present between two layers of fabric that would form the base in this alternative embodiment.

FIGS. 8-11 illustrate another embodiment of the invention. This embodiment is illustrated as having a cover 800 sewn onto the harness 850. The cover 800 includes fabric (for example, a ribbon) having a width wider than the harness strap 860 and a length in excess of the hook section 862 (or loop section 864) of the strap 860. The cover 800 is attached by stitching 802 the cover 800 onto the hook section 862 with a stitch pattern approximately framing the edge of the hook section 862; however, the cover 800 may include its non-free end being stitched and/or attached across its entire width to the strap 860 as illustrated in FIG. 8. The cover 800 in at least one embodiment has its free end 804 looped over and stitched near the end of the strap 860. This orientation of the cover 800 protects the edges of the hook/loop section 862/864 of the strap 860 from being in contact with the infant. One of ordinary skill in the art will appreciate based on this disclosure that there are other ways to attach the cover to the strap including but not limited to adhesives, fusible web, and like attachment means.

In an alternative embodiment, the free end 804 includes a section of hoops or loops to attach to the expose loop 864 or hook section 862 of the strap 860. This embodiment is particularly useful on the straps 880 used to anchor the stir-ups by wrapping around the leg of the infant where a portion of the hooks/loops may not be covered.

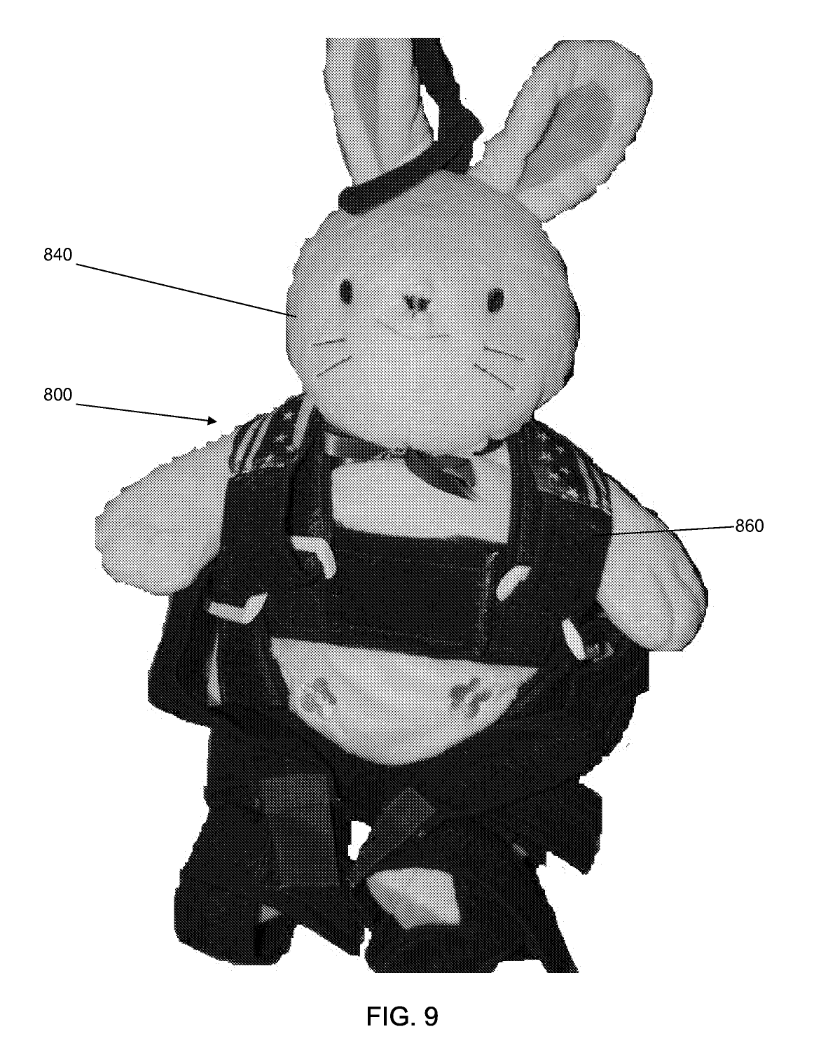

FIGS. 9-11 illustrate the cover shown in FIG. 8 being attached and used on the shoulder section of the harness 850. As illustrated in FIG. 11, the folded over free end 804 of the cover 800 hangs on the back side of the infant (as represented by the stuff bunny 840). As illustrated, the free end 804 provides a grab point for releasing the hook section 862 of the strap 860 from the loop section 864 of the strap 860 and minimizes fraying during washing as well as when putting on and removing the harness on the infant on a daily basis (after the parents have received instruction).

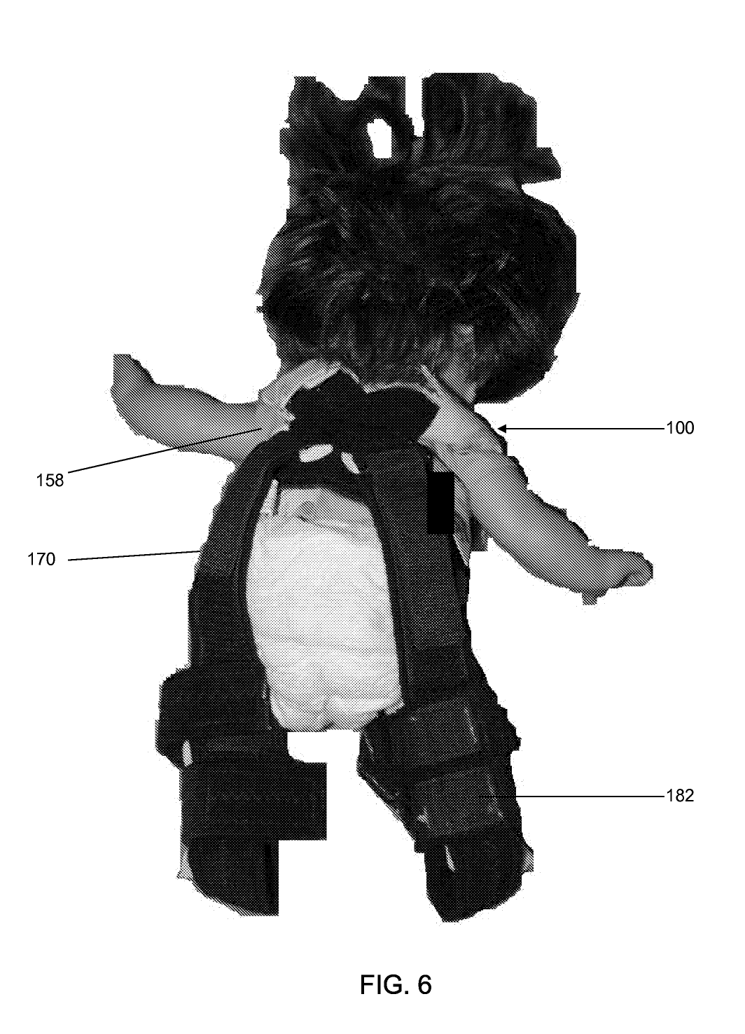

At least one embodiment of the invention includes the harness combined with the above-described embodiments. As illustrated, for example, in FIGS. 3 and 6, the harness 150 includes a chest belt 156 having a pair of shoulder buckles (or rings) 157, four foot buckles (or rings) 158, and two should straps 160. Although the shoulder straps 160 are illustrated in FIG. 6 as extending from the back side of the chest belt 156 and the shoulder buckles 157 are attached on the front of the chest belt 156, these positions may be reversed in an alternative embodiment. The four foot buckles 158 are spaced about the chest belt 156 to attach to leg straps 170. The illustrated harness 150 also includes a pair of foot stir-ups 180 each having a leg strap 170 on the on right and left side of the stir-up 180. Based on this disclosure, one of ordinary skill in the art should appreciate that the foot buckles 158 and attached ends of the leg straps 170 may be reversed in an alternative embodiment. The stir-up 180 also includes a plurality of wrap straps 182 that go around the infant's leg. As discussed previously, the straps 160, 170, 182 include a hook section and a loop section near the free end of the strap 160, 170, 182 (as illustrated, for example, in FIG. 8) such that the strap 160, 170, 182 1) passes through a respective buckle 157, 158 and is looped back on itself to secure the strap 160, 170, 182 in place or 2) the strap 182 attaches to a respective hook/loop section on the back side of the foot stir-ups 180.

As illustrated in FIG. 9, the stitched cover 800 is attached to the shoulder straps 860. In an alternative embodiment, the stitched cover 800 is attached to any combination of straps present in the harness. Likewise, although the fastener cover 100 is illustrated as being located on the shoulder straps 160, it too could be used on the leg straps 170 in the harness 150.

In an alternative embodiment, the fastener cover 100 is directly attached to the strap 160, 170 it is protecting along the face of the strap opposite the hook or loop section farthest from the free end. The base 110 of the cover 100 may be attached with adhesive, sewn on, fasteners, etc. along part or all of its length. These different ways of attaching the cover 100 to the strap 160, 170 can be group together as means for attaching. The unfinished side 116 of the base 110 is the face that abuts the strap 160, 170. The base 110 extends out from the strap 160, 170 on either side of the strap 160, 170 such that the free edges are the edges on which the fastening means are placed. However, in a further alternative embodiment, the base 100 is attached such that it extends beyond just one side of the strap and along the edge that is closest to a strap edge are the edges that the fastening means are placed.

In a further embodiment, a modified onesie is worn by the infant below the harness. The modified onesie includes two pieces including a front piece and a back piece that are connected by the fastening means. Each piece is slide between the harness and the infant's skin and then the two pieces are attached together with the fastening means described previously.

The inventor found that after using the covers 100, 800 illustrated in the figures, cuts that were present on her baby healed up and did not reoccur as the sharp edges that were previously irritating the baby's skin were covered. The use of the covers also reduced skin irritations and abrasions to the breastfeeding mother and other caregivers. The use of covers was found to also reduce the amount of debris collected by the exposed hook sections along with a reduction in the likelihood of having the harness attaching itself to clothing or other fabric.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

Although the present invention has been described in terms of particular example embodiments, it is not limited to those embodiments. The embodiments, examples, and modifications which would still be encompassed by the invention may be made by those skilled in the art, particularly in light of the foregoing teachings.

As used above “substantially,” “generally,” “approximately,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

I claim:

1. A cover for use with a harness for treating developmental dysplasia of the hip, the harness having a chest belt with a pair of shoulder straps and buckles with each shoulder strap having loops and hooks, said cover comprising:
a base with a front face, a back face, and two opposing edges;
a plurality of snap pairs having a socket and a stud, one of each component of said snap pairs is attached to said front face along one edge of said base and the other components of said snap pairs are attached to said rear face along the opposing edge of said base, said snap components are distributed along each edge such that each aligned pair includes one socket and one stud; and
wherein said base having sufficient width between the opposing edges to encircle one shoulder strap when spaced apart hooks and loops engage each other.

2. The cover according to claim 1, wherein said plurality of snap pairs numbers from 2 to 6.

3. The cover according to claim 1, wherein said plurality of snap pairs numbers 3.

4. The cover according to claim 1, wherein said plurality of snap pairs numbers 4.

5. The cover according to claim 1, wherein the snaps are spaced from each other by at least 1.5 inches.

6. The cover according to claim 1, wherein there are covers for each strap in the harness having one loop section and one hook section.

7. The cover according to claim 1, wherein the base has a width of between 4.00 and 4.25 inches and a length of between 4.50 and 6.25 inches.

8. The cover according to claim 1, wherein said base is made of soft material.

9. A harness for treating developmental dysplasia of the hip, said harness comprising:
a chest belt;
at least two chest buckles attached directly to said chest belt and spaced from each other; and
at least two shoulder straps extending from said chest belt, each shoulder strap capable of passing through one of said chest buckles, each shoulder strap having
a free end with a hook section and a loop section,
a cover attached to at least one of said hook section and said loop section, said cover extending beyond at least one of said free end and sides of said hook section and said loop section, and said cover having a width wider than at least one of said hook section and said loop section.

10. The harness according to claim 9, further comprising a pair of foot attachments, wherein
each foot attachment having a pair of foot buckles,
said chest belt having
a posterior leg strap,
an anterior leg strap, and
each leg strap capable of passing through one of said foot buckles and having
a free end with a hook section and a loop section,
a cover attached to at least one of said hook section and said loop section, said cover extending beyond said free end and having a width wider than at least one of said hook section and said loop section.

11. The harness according to claim 9, wherein said cover is attached to said strap spaced from the free end of said strap, said cover having a front face, a back face, and two opposing edges spaced from said strap, means for fastening said front face to said back face along respective opposing edges and around the strap when the strap is folded back on itself through one buckle.

12. The harness according to claim 11, further comprising a pair of foot attachments, wherein
each foot attachment having a pair of foot buckles,
said chest belt having
a posterior leg strap,
an anterior leg strap, and
each leg strap capable of passing through one of said foot buckles and having
a free end with a hook section and a loop section,
a cover attached to at least one of said hook section and said loop section, said cover extending beyond said free end and having a width wider than at least one of said hook section and said loop section.

13. The harness according to claim 11, further comprising a pair of foot attachments, and wherein
said chest belt includes a plurality of foot buckles,
each foot attachment having
a posterior leg strap,
an anterior leg strap, and
each leg strap capable of passing through one of said foot buckles and having
a free end with a hook section and a loop section,
a leg strap cover attached to at least one of said hook section and
said loop section, said leg strap cover extending beyond the sides of said strap, said leg strap cover having a front face, a back face, and two opposing edges spaced from said strap, means for fastening said front face to said back face along respective opposing edges and around the hook section and the loop section when the hook and loop section are engaging each other.

14. The cover according to claim 11, wherein said means for fastening includes at least one of snaps, buttons, hook and loop circle tabs, and plastic hook enclosures.

15. The cover according to claim 11, wherein said means for fastening includes a plurality of snap pairs numbers from 2 to 6.

16. The cover according to claim 15, wherein the snaps are spaced from each other by at least 1.5 inches.

17. The cover according to claim 11, wherein the cover has a width of between 4.00 and 4.25 inches and a length of between 4.50 and 6.25 inches.

18. A cover for use with a harness for treating developmental dysplasia of the hip, the harness having a chest belt with a pair of shoulder straps and buckles with each shoulder strap having hooks and loops, said cover comprising:
a base with a front face, a back face, and two opposing edges;
a plurality of means for fastening said front face to said back face along their respective opposing edges; and
wherein said base having sufficient width between the opposing edges to encircle one shoulder strap when it is doubled back on itself through one buckle.

19. The cover according to claim 18, wherein said means for fastening includes at least one of snaps, buttons, hook and loop circle tabs, and plastic hook enclosures.

20. The cover according to claim 18, wherein the base has a width of between 4.00 and 4.25 inches and a length of between 4.50 and 6.25 inches.
